(19) United States
(12) Patent Application Publication
(10) Pub. No.: US 2011/0288568 A1
(43) Pub. Date: Nov. 24, 2011

(54) IMPLANTABLE PROSTHESIS

(76) Inventors: Thomas J. Capuzziello, Milford, MA (US); Marianne Staudenmeier, North Kingstown, RI (US); Michael A. Cote, Lincoln, RI (US); Brian Leach, Cranston, RI (US)

(21) Appl. No.: 13/140,556

(22) PCT Filed: Dec. 19, 2008
(86) PCT No.: PCT/US08/13910

§ 371 Date: Aug. 16, 2011

(51) Int. Cl. A61B 17/00

(52) U.S. Cl. 606/151

(57) ABSTRACT

An implantable prosthesis for repairing a tissue or muscle wall defect. The prosthesis includes a perforated sheet of a biologically compatible material to cover the tissue or muscle wall defect. The perforated sheet has a plurality of perforations extending through the sheet. The perforations may be distributed across the perforated sheet in a non-grid arrangement. The perforations may be non-uniformly distributed across the perforated sheet in groups of perforations arranged in a plurality of concentric circular patterns. Each perforation may be separated from an adjacent perforation by a web of material having a length between adjacent perforations that is a predetermined multiple of the diameter of the perforations.

### BACKGROUND OF INVENTION

1. Field of Invention

The present invention relates to an implantable prosthesis, and methods for using such a prosthesis, for repairing, reconstructing, buttressing, or augmenting soft tissue or muscle wall defects.

2. Discussion of Related Art

Various prosthetic repair materials are known for repairing and reinforcing anatomical defects, such as soft tissue and muscle wall hernias. For example, ventral and inguinal hernias are commonly repaired using a sheet of biocompatible fabric, such as a knitted polypropylene mesh (e.g., BARD MESH). Once inserted into a patient, the fabric is typically sutured, stapled, tacked or otherwise provisionally anchored in place over, under or within the defect. Tissue integration with the fabric, such as by tissue ingrowth into the fabric, eventually completes the repair.

Other types of prosthetic repair material used for hernia repair may be derived from natural material. For example, hernias may be repaired using a sheet of material derived from porcine dermis (e.g., BARD COLLAMEND).

A known prosthetic repair material, such as illustrated in FIG. 1, includes a sheet 10 of either natural or synthetic material having a plurality of perforations 12. As shown, the perforations 12 are uniformly distributed in a series of rows 14 and columns 16 across the sheet, thereby forming a grid arrangement of perforations.

There remains a need for improved an implantable prosthesis that is suitable for repairing, reconstructing and/or augmenting defects or weaknesses in tissues and organs.

### SUMMARY OF INVENTION

The present invention relates to a prosthetic device for repairing an anatomical defect or weakness, such as a tissue or muscle wall hernia.

In one embodiment, an implantable prosthesis is provided for repairing or augmenting a tissue or muscle wall defect. The implantable prosthesis includes a perforated sheet of a biologically compatible material to cover the tissue or muscle wall defect. The perforated sheet has a plurality of perforations extending completely through the perforated sheet. The perforations are distributed across a substantial portion of the perforated sheet in a non-grid arrangement. Each of the plurality of perforations has a diameter, and each perforation is separated from an adjacent perforation by a portion of material having a length between adjacent perforations that is at least four times the diameter of the perforations.

In another embodiment, an implantable prosthesis is provided for repairing or augmenting a tissue or muscle wall defect. The implantable prosthesis includes a perforated sheet of a biologically compatible material to cover the tissue or muscle wall defect. The perforated sheet has a plurality of perforations extending completely through the perforated sheet. The perforations are non-uniformly distributed across the perforated sheet in an arrangement that includes groups of perforations arranged in a plurality of concentric circular patterns about a reference point. Each of the plurality of perforations has a diameter, and the plurality of concentric circular patterns are spaced apart from each other by a radial distance relative to the reference point of at least four times the diameter of the perforations.

In yet another embodiment, an implantable prosthesis is provided for repairing or augmenting a tissue or muscle wall defect. The implantable prosthesis includes a perforated sheet of a biologically compatible material to cover the tissue or muscle wall defect. The perforated sheet has a plurality of perforations extending completely through the perforated sheet. The perforated sheet includes a first portion and a second portion separated by an imaginary straight line extending across the perforated sheet and through the center of the sheet. The plurality of perforations are non-uniformly distributed across a substantial portion of the perforated sheet, including a first pattern of perforations in the first portion of the perforated sheet and a second pattern of perforations in the second portion of the perforated sheet, where the second pattern is different from the first pattern.

In a further embodiment, an implantable prosthesis is provided for repairing or augmenting a tissue or muscle wall defect. The implantable prosthesis includes a perforated sheet of a biologically compatible material to cover the tissue or muscle wall defect. The perforated sheet has a plurality of perforations extending completely through the perforated sheet. Each of the plurality of perforations has a diameter, and each perforation is separated from an adjacent perforation by a portion of material having a length between adjacent perforations that is at least four times the diameter of the perforations. The perforations are distributed across the perforated sheet in an arrangement that includes groups of perforations arranged in a plurality of concentric circular patterns about a reference point, with the plurality of concentric circular patterns radially spaced apart from each other by a substantially equal distance.

In another embodiment, a method is provided of repairing or augmenting a tissue, muscle or organ defect with an implantable prosthesis including a perforated sheet of a biologically compatible material to cover the tissue, muscle or organ defect. The perforated sheet has a plurality of perforations extending completely through the sheet, with the perforations being distributed across a substantial portion of the perforated sheet in a non-grid arrangement. Each of the plurality of perforations has a diameter, with each perforation being separated from an adjacent perforation by a portion of material having a length between adjacent perforations that is at least four times the diameter of the perforations. The method involves positioning the implantable prosthesis so that the perforated sheet covers a defect, and securing the perforated sheet in place relative to the defect.

Various embodiments of the present invention provide certain advantages. Not all embodiments of the invention share the same advantages and those that do may not share them under all circumstances.

Further features and advantages of the present invention, as well as the structure of various embodiments of the present invention are described in detail below with reference to the accompanying drawings.

### BRIEF DESCRIPTION OF DRAWINGS

In the drawings, each identical or nearly identical component that is illustrated in the various figures is represented by a like numeral. For purposes of clarity, not every component may be labeled in every drawing.

Various embodiments of the invention will now be described, by way of example, with reference to the accompanying drawings, in which:

FIG. 1 is a plan view of a known implantable prosthesis;

FIG. 2 is a plan view of an implantable prosthesis according to one illustrative embodiment;

FIG. 2A is a representative cross-sectional view of the prosthesis illustrated in FIG. 2 taken along section line 2A-2A;

FIG. 3 is a plan view of an implantable prosthesis according to another illustrative embodiment;

FIG. 4 is a plan view of an implantable prosthesis according to a further illustrative embodiment;

FIG. 5 is a plan view of an implantable prosthesis according to another illustrative embodiment; and

FIG. 6A-6H illustrate various perforation patterns of implantable prostheses according to other illustrative embodiments.

### DETAILED DESCRIPTION

The invention is directed to an implantable prosthesis for repairing or augmenting anatomical defects or weaknesses, and is particularly suitable for the repair of defects in, and weaknesses of, soft tissue, muscle or organ walls or other anatomical regions. Although the prosthetic device is particularly suited for hernia repair, it should be understood that the prosthesis is not so limited and may be employed to repair various defects and anatomical structures, as would be apparent to one of skill in the art.

The invention is more particularly directed to a prosthesis that includes a sheet of biologically compatible material that is configured to cover or extend across the defect or weakness. In this regard, the material may be larger than at least a portion of the defect or weakness so that placement of the sheet against the defect will cover or extend across that portion of the opening or weakness.

The sheet of material includes a plurality of perforations extending completely through the sheet. The perforations may facilitate tissue or muscle ingrowth to enhance the repair of the defect. In this regard, the perforations may allow sufficient tissue or muscle ingrowth to integrate the prosthesis with host tissue or muscle after implantation.

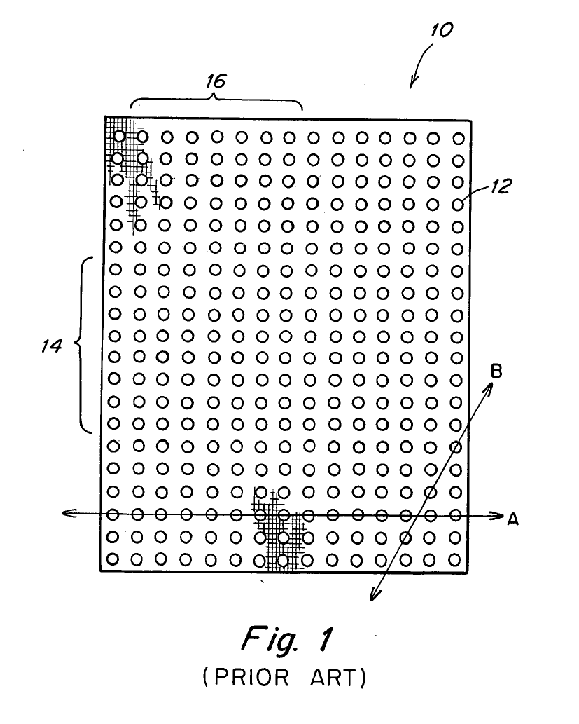

In contrast to the known repair material shown in FIG. 1, the prosthetic device according to one embodiment of the present invention may have a plurality of perforations that are distributed across the sheet of material in a non-grid arrangement. Applicant believes that the non-grid arrangement may provide certain advantages over a grid-like arrangement, such as the arrangement shown in FIG. 1.

Perforations uniformly distributed across the sheet in a grid arrangement may lead to mechanical properties that vary in different directions across the prosthesis. For example, in a grid arrangement, the tensile strength of the prosthesis in one direction along the path of either a row 14 or a column 16 (such as along line A) may vary from the tensile strength of the prosthesis in another direction (such as along line B). Thus, according to one aspect of the invention, perforations may be distributed in a non-grid arrangement in an effort to reduce the variation in mechanical properties of the prosthesis in various directions across the prosthesis.

The perforations may be non-uniformly distributed across a substantial portion or selected portions of the perforated sheet. In one embodiment, the perforations may be distributed in groups of perforations arranged in a plurality of concentric circular patterns about a common reference point. A perforation may be provided at the common reference point. The perforations may be non-uniformly distributed across the perforated sheet such that there is a first pattern of perforations in a first portion of the sheet and a second pattern of perforations in a second portion of the sheet, where the second pattern is different from the first pattern. It should be appreciated that the invention also contemplates other perforation patterns, as it is not so limited.

The prosthesis may be configured in any desired shape suitable for the particular repair. For example, the prosthesis may have a non-circular shape, such as a generally oval, elliptical or egg shape, that is suitable for augmenting or repairing a hernia. The prosthesis may be shaped so as to have a major axis and a minor axis. It should also be recognized that the invention contemplates other shapes, as it is not so limited.

The prosthesis may be composed of either a solid or substantially non-porous material, or it may be formed of a tissue infiltratable material, such as a knit fabric. The prosthesis may be formed of one or more layers of the same or dissimilar material. The prosthesis may be formed with portions that are tissue infiltratable and other portions that are non-tissue infiltratable, providing selected areas of the repair device with different tissue ingrowth and adhesion resistant properties.

The prosthesis may be formed of a natural and/or synthetic material. Some examples of natural materials suitable for the prosthesis include, but are not limited to, collagen materials derived from porcine dermis, porcine small intestine submucosal, porcine pericardium, bovine dermis, bovine small intestine submucosal, or bovine pericardium. Some examples of synthetic materials suitable for the prosthesis include, but are not limited to, polylactic acid (PLA), polyglycolic acid (PGA), expanded polytetrafluorethylene (ePTFE), or polyhyaluronic acid (PHA). The prosthesis may be formed of a resorbable and/or a non-resorbable material.

Turning now to the drawings, it should be appreciated that the drawings illustrate various components and features which may be incorporated into one or more embodiments of the present invention. For simplification, several drawings may illustrate more than one optional feature or component. However, the present invention is not limited to the specific embodiments disclosed in the drawings. It should be recognized that the present invention encompasses one or more embodiments which may include only a portion of the components illustrated in any one figure, and/or may also encompass one or more embodiments combining components illustrated in multiple different drawings, and/or may also encompass one or more embodiments not explicitly disclosed in the drawings.

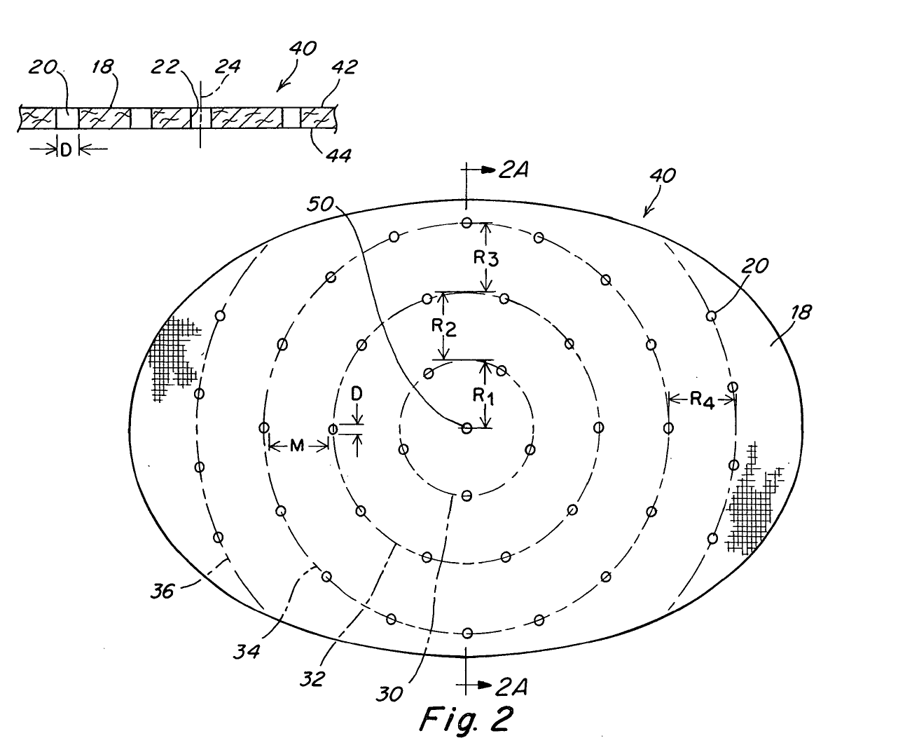

FIG. 2 illustrates one embodiment of a prosthesis 40 that includes a perforated sheet 18 of biologically compatible material that is configured to cover the defect. The prosthesis 40 is configured as a patch that may be used, for example, as an underlay or an overlay for hernia repair. The prosthesis 40 may be configured with any desired strength, flexibility, tissue integration, adhesion resistance and/or other characteristics suitable for the repair, as would be apparent to one of skill in the art. Although the prosthesis 40 is described in connection with a patch-type embodiment, the prosthesis may include a plug, a combination plug and patch, and other suitable arrangements for repairing the defect.

The perforated sheet 18 includes a plurality of perforations 20 which extend completely through the perforated sheet, from a first side 42 to a second side 44 (see FIG. 2A). The perforations 20 may be configured to facilitate tissue or muscle ingrowth to enhance the repair of the defect. It is also contemplated that the perforations 20 may assist with fluid drainage through the sheet.

As mentioned above, and as illustrated in FIG. 2, the perforations 20 may be distributed across a substantial portion of the sheet in a non-grid arrangement. The known grid arrangement illustrated in FIG. 1, includes a series of rows 14 and columns 16 of perforations 12. In contrast, the perforations 20 shown on the perforated sheet in FIG. 2 are not arranged in rows and columns.

Applicant believes that a grid arrangement, such as shown in FIG. 1, may lead to mechanical properties that vary in different directions. As mentioned above, when perforations are distributed in a grid arrangement, mechanical properties, such as the tensile strength, elongation or stretch and/or stiffness, may vary significantly in various directions. This variation in mechanical properties may require particular orientation of the repair material in the body. In contrast, use of a non-grid arrangement of perforations may provide less variation in the mechanical properties in different directions across the prosthesis. Thus, it is contemplated that the more uniform mechanical properties of the prosthesis will enable the prosthesis 40 to be utilized in any angular orientation.

Furthermore, with grid arrangements, such as shown in FIG. 1, the rows 14 and columns 16 of perforations may lead to relatively weaker areas along which the prosthesis 10 may potentially tear or stretch, such as along either a row 14 and/or a column 16. For example, a small tear or weakened area may be more likely to propagate in the direction of a row 14 and/or column 16. In contrast, in a non-grid arrangement of the present invention, the perforations 20 may be arranged so that they do not form a linear pattern, such that a tear may be less likely to propagate through the prosthesis.

The perforations 20 may have a substantially circular shape with a diameter D. In one embodiment, as illustrated in FIG. 2, the diameter D of the perforations 20 is substantially constant. However, it should be appreciated that other embodiments may employ perforations with diameters that may vary from each other, as the invention is not so limited. Furthermore, in other embodiments, the perforations may be shaped differently. For example, the perforations may be square shaped, rectangular shaped, triangular shaped, oval shaped and/or irregular shaped, as the invention is not so limited. It should be recognized that the term “diameter” is used to broadly define the width of the perforation 20, and the term “diameter’ may broadly be used to define the width of perforations having other shapes, as the invention is not so limited.

In one embodiment, as illustrated in FIG. 2A, the perforations 20 extend completely through the sheet 18 such that the walls 22 defining the perforation 20 are substantially perpendicular to the first side 42 of the sheet and/or the second side 44 of the perforated sheet. As shown, an axis 24 defining the angular orientation of the perforation is substantially perpendicular to the first and second sides 42, 44 of the sheet. It should be appreciated that, in other embodiments, the perforations may be configured differently, as the invention is not so limited. For example, it is also contemplated that the perforations 20 may be tapered such that the diameter D of a perforation on the first side 42 is greater than the diameter of the same perforation on the second side 44.

In one embodiment, the perforations 20 are circular and have a diameter D of approximately 0.028 inches to 0.157 inches. In one embodiment, the diameter of the perforations 20 is at least 0.094 inches (˜2.3 mm). In another embodiment, the diameter of the perforations 20 is at least 0.125 inches. However, it is to be appreciated that the perforations may be configured in other suitable shapes and sizes as would be apparent to one of skill in the art.

It may be desirable to maximize the number of perforations 20 distributed across the perforated sheet 18 to facilitate tissue ingrowth into the prosthesis 40. In one embodiment, such as shown in FIG. 2, the perforations 20 may be distributed across a substantial portion of the sheet.

The number of perforations 20 on the sheet 18 may affect certain mechanical properties of the prosthesis. For example, an increased number of perforations may lower the tensile strength of the prosthesis and/or increase the elongation or stretch of the sheet. Thus, it may be desirable to maintain a minimum spacing between adjacent perforations 20, to provide certain desirable mechanical properties of the sheet 18.

The perforations spacing may be based on the size of the perforations. The web ratio may be defined as the ratio of the length of material between adjacent perforations divided by the diameter of the perforations. In one embodiment, the perforations 20 are distributed across the sheet such that the each perforation is separated from an adjacent perforation by a web of material M having a length between adjacent perforations that is at least equal to the diameter D of the perforations 20 (web ratio of 1:1). In another embodiment, the perforations are distributed across the sheet such that each perforation is separated from an adjacent perforation by a web of material M having a length that is at least two times the diameter D of the perforations 20 (web ratio of 2:1). In yet another embodiment, adjacent perforations are spaced apart from each other by a web of material M that is at least three times the diameter of the perforations (web ratio of 3:1). In yet another embodiment, the web of material M between adjacent perforations 20 is at least four times the diameter D of the material (web ratio of 4:1). In one illustrative embodiment as shown in FIG. 2, adjacent perforations 20 are spaced apart from each other by a web of material M having a length that is at least five times the diameter D of the material (web ratio of 5:1). It is to be understood that the prosthesis may employ other web ratios for perforation spacing as would be apparent to one of skill in the art.

The perforations 20 may be non-uniformly distributed across the perforated sheet 18 in an arrangement that includes groups of perforations. In one illustrative embodiment shown in FIG. 2, the perforations are arranged in a plurality of concentric circular patterns 30, 32, 34, 36 about a common reference point 50. A perforation may be provided at the reference point. As illustrated, the reference point 50 may be located substantially at the center of the perforated sheet 18. However, it should be appreciated that in other embodiments, the reference point 50 may be located off-center, as the invention is not so limited.

The plurality of concentric circular patterns 30, 32, 34, 36 may be spaced apart from each other by a minimum radial distance R₁, R₂, R₃, R₄ relative to the reference point 50. Arranging the concentric circular patterns with a minimum radial distance therebetween may help to maintain a desired minimum spacing between adjacent perforations 20, to thus achieve desirable mechanical properties of the sheet 18.

The radial distance between the concentric circular patterns may be based on the size of the perforations. In one embodiment, the minimum radial distance R_{I}, R₂, R₃, R₄ between the concentric circular patterns 30, 32, 34, 36 relative to the reference point 50 is at least equal to the diameter D of the perforations 20. In another embodiment, the minimum radial distance between the concentric circular patterns relative to the reference point is at least two times the diameter of the perforations 20. In another embodiment, the minimum radial distance between the concentric circular patterns is at three times the diameter of the perforations 20. In yet another embodiment, the minimum radial distance R₄ between the concentric circular patterns is at least four times the diameter of the perforations 20. In one illustrative embodiment as shown in FIG. 2, the minimum radial distance R_{I}, R₂, R₃, R₄ between the concentric circular patterns 30, 32, 34, 36 relative to the reference point 50 is at least five times the diameter D of the perforations 20. However, it is to be understood that the prosthesis may employ other radial distances between the concentric circular patterns as would be apparent to one of skill in the art.

The concentric circular patterns 30, 32, 34, 36 may be radially spaced apart from each other by a substantially equal distance. In this regard, in one embodiment, R₁=R₂=R₃=R₄. In one illustrative embodiment, each circular pattern 30, 32, 34, 36 is radially spaced apart from each other by at least 0.5 inches. In other embodiments, the concentric circular patterns may be radially spaced apart from each other by different distances, as the invention is not so limited.

The size of the concentric circular patterns 30, 32, 34, 36 may vary. In one illustrative embodiment as shown in FIG. 2, the diameter of the first circular pattern 30 is approximately 1.19 inches, the diameter of the second circular pattern 32 is approximately 2.38 inches, the diameter of the third circular pattern 34 is approximately 3.56 inches, and the diameter of the fourth circular pattern 36 is approximately 4.75 inches. In one illustrative embodiment, the implantable prosthesis 40 is approximately 6 inches long and 4 inches wide. As shown in FIG. 2, the fourth circular pattern 36 extends across only a portion of the prosthesis 40 because the diameter of the fourth circular pattern 36 is greater than the width of the prosthesis 40.

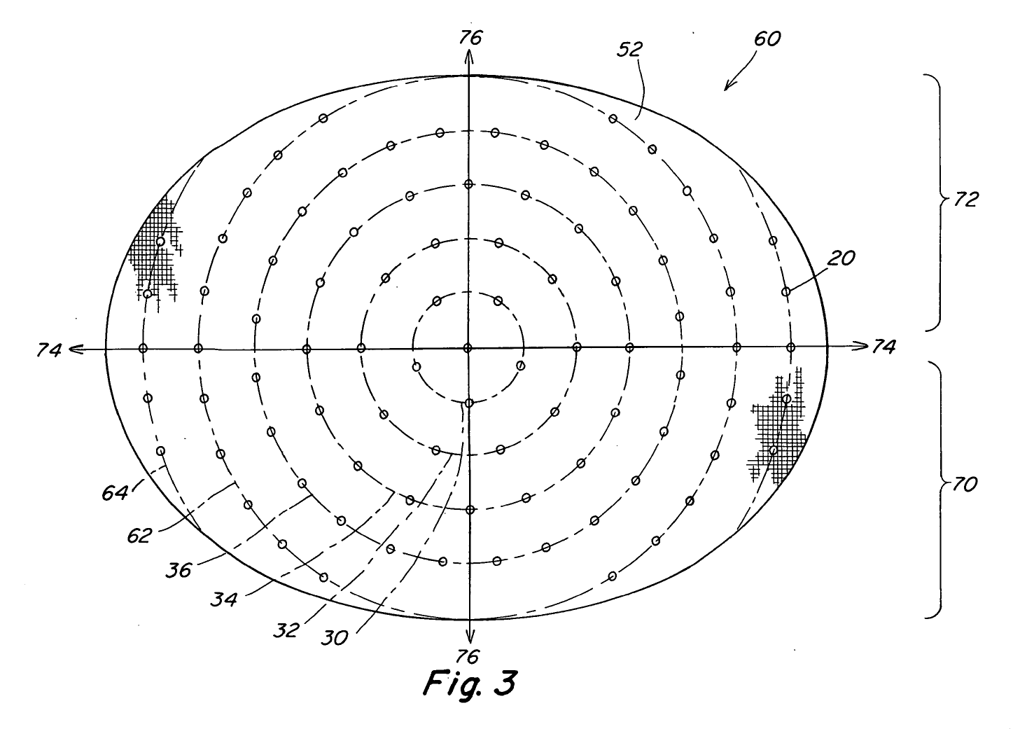

In another illustrative embodiment as shown in FIG. 3, a prosthesis 60 includes a perforated sheet 52 of biologically compatible material that is configured to cover the defect. This embodiment is similar to the embodiment illustrated in FIG. 2, except that it is larger and has a greater number of concentric circular patterns. In one embodiment, the implantable prosthesis 60 is approximately 8 inches long and 6 inches wide, with the inner four circular patterns 30, 32, 34, 36 being substantially the same as those shown in FIG. 2. As illustrated in FIG. 3, the prosthesis 60 includes a fifth circular pattern 62 and a sixth circular pattern 64 that are concentric with the inner four circular patterns 30, 32, 34, 36.

As indicated above, the perforations 20 may be non-uniformly distributed across the sheet such that there are a plurality of different perforation patterns. This is in contrast to a sheet having perforations arranged in a grid arrangement, such as shown in FIG. 1, where there is only a single perforation pattern. In the illustrative embodiment of FIG. 3, the perforated sheet 52 may include a first portion 70 and a second portion 72 located on opposing sides of an imaginary straight line 74 extending across the perforated sheet 52 and through the center of the sheet. The perforations 20 in the first portion 70 are distributed in a first pattern and the perforations 20 in the second portion 72 are distributed in a second pattern. As illustrated, the second pattern is different from the first pattern. In this respect, the perforations 20 may be distributed across the sheet such that the first and second patterns are not symmetrical about the straight line 74 extending through the perforated sheet 52 and through the center of the sheet.

The perforations 20 may also be non-uniformly distributed across the sheet such that there are a plurality of different perforation patterns that are symmetrical about a straight line extending across the perforated sheet 52 through the center of the sheet. For example, as illustrated in FIG. 3, the perforation pattern is symmetrical about a straight line 76.

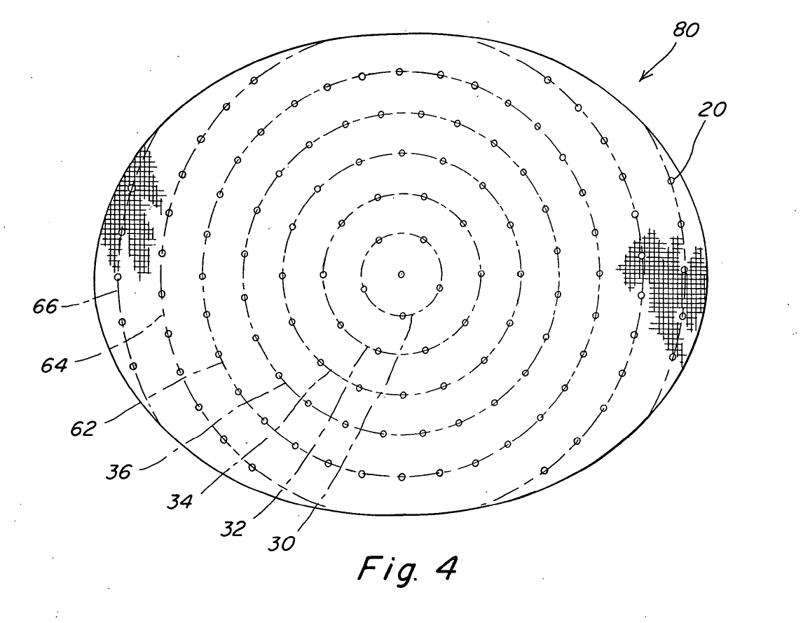

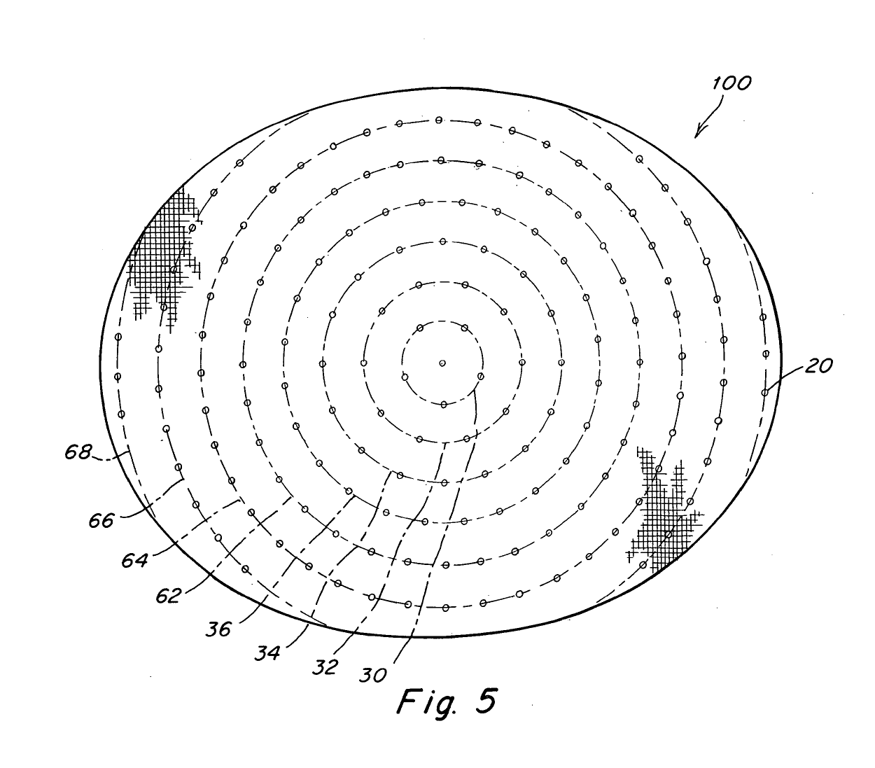

In other illustrative embodiments as shown in FIGS. 4 and 5, the prostheses 80, 100 include a perforated sheet of biologically compatible material that is configured to cover the defect. These prostheses are similar to the embodiments illustrated in FIGS. 2 and 3, except that each prosthesis is larger and includes a greater number of concentric circular patterns. In the embodiment illustrated in FIG. 4, the prosthesis 80 includes a seventh circular pattern 66 of perforations in addition to the six circular patterns 30, 32, 34, 36, 62, 64 of FIG. 3. The implantable prosthesis 80 is approximately 9 inches long and 7 inches wide. In the embodiment illustrated in FIG. 5, the prosthesis 100 includes an eighth circular pattern 68 in addition to the perforations shown in FIG. 4. The prosthesis 100 is approximately 10 inches long and 8 inches wide.

The implantable prosthesis 40 may be formed from a variety of different materials, as the invention is not limited in this respect. In one illustrative embodiment, the prosthesis 40 is derived from a natural material, such as a collagen material. In one embodiment, the prosthesis is derived from porcine dermis. It is also contemplated that the sheet of material may be derived from porcine small intestine submucosal, porcine pericardium, bovine dermis, bovine small intestine submucosal, or bovine pericardium. However, it is to be understood that the prosthesis may be formed of other suitable materials apparent to one of skill in the art.

In one embodiment, the sheet of material is a collagen material that is prepared according to the techniques disclosed in U.S. application Ser. No. 11/508,438, which is herein incorporated by reference in its entirety. As set forth in greater detail in the '438 application, the prosthesis 40 may be crosslinked with a crosslinking agent. Crosslinking includes treatment of the treated (acellular) tissue with a crosslinking agent to stabilize the tissue such that it resists enzymatic degradation (i.e., resorption), to impart increased strength and to provide structural integrity to the implant.

In one embodiment, the implantable prosthesis 40 is made with a sheet of material formed from crosslinked porcine dermis, such as BARD COLLAMEND, available from C. R. Bard, Inc. of Murray Hill, N.J. In this embodiment, the prosthesis 40 is formed from a sheet of lyophilized, acellular porcine dermal collagen and its constituent elastin fibers. The sheet is processed to remove non-collagenous cellular components and the sheet is crosslinked to increase the strength and the time period for resorption.

The amount of material is crosslinking controls the time period for resorption. In general, increasing the amount of crosslinking will increase the period of time for the material to completely resorb. As mentioned above, in one embodiment, the sheet of material forming the implantable prosthesis is resorbable. In one particular embodiment, the material is configured to be completely resorbed more than one year after the material is implanted into a body. In another embodiment, the material is configured to be completely resorbed more than two years after the material is implanted into the body. It should be appreciated that the time period for resorption of the prosthesis may be varied by altering the amount the material is crosslinked as would be apparent to one of skill in the art.

In one embodiment, the implantable prosthesis may be formed with a synthetic material. Examples of suitable synthetic materials include, but not limited to, polylactic acid (PLA), polyglycolic acid (PGA), expanded polytetrafluorethylene (ePTFE), or polyhyaluronic acid (PHA). It is also contemplated that the prosthesis 40 may include both natural and synthetic material, as the invention is not so limited. The synthetic material may either be a resorbable or non-resorbable material.

In one embodiment, the thickness of the prosthesis 40 may range from about 0.8 mm to about 1.3 mm. In one embodiment, the thickness of the prosthesis is about 1 mm. With a sheet of material derived from porcine dermis, the source material of the porcine dermis layer may have an initial thickness of approximately 2 mm-2.5 mm. Material may be removed from each side of the source material to obtain the desired collagen from the middle portion of the layer. In one embodiment, each side of the source material is thinly sliced to remove material from each side. It should be appreciated that the thickness of the prosthesis 40 may vary based upon the particular embodiment.

The prosthesis may be configured to have any suitable shape or size that is conducive to facilitating the correction or repair of a particular defect, such as a hernia. As shown in FIG. 2A, the prosthesis 40 has a relatively flat configuration. However, the prosthesis 40 need not be flat, and convex, concave, convex/concave, and more complex three-dimensional shapes also are contemplated. The prosthesis 40 may be pliable to facilitate manipulation and/or reduction of the patch during delivery to the defect and/or to conform the patch to the anatomical site of interest.

In the illustrative embodiments shown in FIGS. 2-5, the prosthesis has a generally oval, elliptical or egg shape suitable for augmenting or repairing an inguinal hernia and/or a ventral hernia. The geometry of the prosthesis 40 is generally non-circular or elliptical with a major axis 74 (FIG. 3) extending along the longest portion of the prosthesis and a minor axis 76 (FIG. 3) extending across the widest portion of the prosthesis in a direction perpendicular to the major axis. It is to be appreciated that the prosthesis may be configured with any suitable shape, such as a shape that is symmetric about both axes, asymmetric about both axes, or asymmetric about the major axis and symmetric about the minor axis. Examples of other shapes include, but are not limited to, circular, square, rectangular, triangular, and irregular configurations. The prosthesis may be sized to cover part or, preferably, all of the defect. Furthermore, it should be appreciated that the implantable prosthesis may be configured such that a user can modify the outer shape of the prosthesis, such as, for example, by trimming the sheet into a desired shape, based on the particular application.

The perforations 20 may be formed in the sheet of material with a mechanical punching device. However, it is contemplated that the perforations may be formed into the material using other techniques apparent to one of skill, as the invention is not so limited.

The prosthesis may be placed at the defect site using an open surgical procedure, or by laparoscopically passing the device through a cannula to the defect. The prosthesis may be flexible, allowing for reduction of the prosthesis, such as by folding, rolling or otherwise collapsing the prosthesis, into a slender configuration suitable for delivery to the defect site. Upon delivery, the prosthesis may automatically open to an unfurled or spread out configuration, or may be unfolded, unrolled or otherwise deployed by the surgeon to an unfurled or spread out configuration suitable to repair the weakness or defect.

In some applications, the prosthesis 40 may be used for tension free repair of a defect without pulling tissue and/or muscle together under tension. In one application, fasteners may be placed about the periphery of the prosthesis 40 to secure the prosthesis to the body. In one embodiment, sutures may be placed about the periphery of the prosthesis 40 and spaced approximately 1-3 cm apart. However, it is to be other suitable fasteners, such as staples or adhesive, may be employed to secure the prosthesis relative to the defect as would be apparent to one of skill in the art.

### Examples

The following examples are illustrative only and are not intended to limit the scope of the present invention.

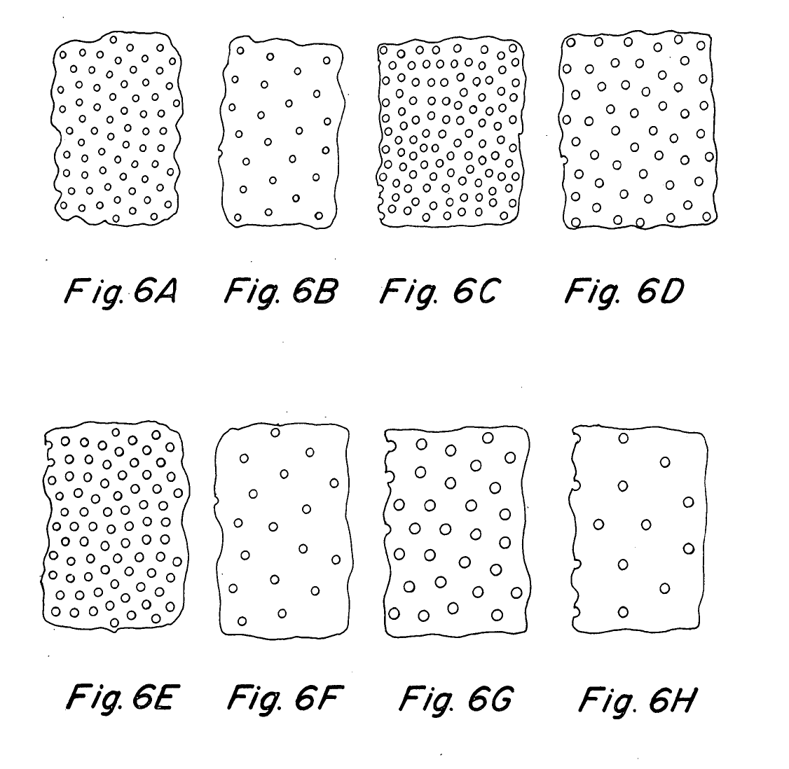

Mechanical properties of eight different perforated implantable prostheses having different perforation patterns were tested and the resulting data is illustrated in Tables 1-6 below. Each of the eight implantable prostheses were made from a material derived from porcine dermis. A representative portion of the perforation pattern for each of the eight prostheses is illustrated in FIGS. 6A-6H. Mechanical properties were tested including thickness, suture pull out strength, burst strength, tear strength, tensile strength, and stiffness.

Table 1 below sets forth the diameter of the perforations and web ratio for each of the eight embodiments of FIGS. 6A-6H. The web ratio is the ratio of the minimum length of material between adjacent perforations divided by the diameter of the perforations. Patterns 1, 2 and 5-8 include groups of perforations non-uniformly arranged in a plurality of concentric circular patterns about a reference point. Patterns 3 and 4 include perforations arranged in randomized patterns.

TABLE 1

| Perforation | Perforation Diameter | | Web |
|---|---|---|---|
| Pattern # | (inches) | Type of Pattern | Ratio |
| 1 | 0.094 | Circular Pattern | 2 to 1 |
| 2 | 0.094 | Circular Pattern | 4 to 1 |
| 3 | 0.125 | Random Pattern | 1 to 1 |
| 4 | 0.125 | Random Pattern | 4 to 1 |
| 5 | 0.125 | Circular Pattern | 1 to 1 |
| 6 | 0.125 | Circular Pattern | 4 to 1 |
| 7 | 0.156 | Circular Pattern | 2 to 1 |
| 8 | 0.156 | Circular Pattern | 4 to 1 |

Sample Thickness: A sample of material was measured in the dry state using a standard thickness snap gage with an approximate 0.375 inch diameter pressure foot that is lightly spring loaded. The thickness was measured by lowering the foot onto the material. Measurements were taken at fifteen locations across the sample of material and then averaged. Thickness data is provided in Table 2, measured in millimeters.

TABLE 2

| | Pattern | | | | | | | |
|---|---|---|---|---|---|---|---|---|
| | 1 | 2 | 3 | 4 | 5 | 6 | 7 | 8 |
| Mean | 1.23 | 1.24 | 1.17 | 1.21 | 1.07 | 1.19 | 1.14 | 1.25 |
| Std. Dev. | 0.11 | 0.08 | 0.10 | 0.08 | 0.10 | 0.10 | 0.11 | 0.06 |
| N | 15 | 15 | 15 | 15 | 15 | 15 | 15 | 15 |
| Min. | 1.05 | 1,13 | 0.98 | 1.07 | 0.91 | 1.01 | 0.94 | 1.12 |
| Max. | 1.39 | 1.39 | 1.33 | 1.3 | 1.28 | 1.3 | 1.3 | 1.32 |

Suture Pullout Strength: A sample of material was prepared and clamped in the lower jaw of a tensile test machine. At least 1 inch of the material was exposed above the jaw. A spring steel wire with a diameter of approximately 0.019 inches was placed through the sample to simulate a suture. The sample was trimmed 4±0.2 mm from the edge of a perforation and the wire was placed through the edge of the perforation so that the sample was tested with a 4 mm bite. The wire suture was looped back and both ends were attached to the upper jaw of the tensile machine. The suture was then pulled at a rate of 5 inches per minute through the sample starting with a minimum jaw separation of 1 inch. The peak force was recorded for fifteen samples. Suture pullout strength data is provided in Table 3, measured in pound-force (lbf).

TABLE 3

| | Pattern | | | | | | | |
|---|---|---|---|---|---|---|---|---|
| | 1 | 2 | 3 | 4 | 5 | 6 | 7 | 8 |
| Mean | 8.24 | 7.6 | 8.24 | 8.21 | 8.85 | 9.09 | 7.87 | 10.28 |
| Std. Dev. | 2.37 | 2.23 | 1.85 | 2.54 | 1.84 | 1.92 | 2.24 | 1.58 |
| N | 15 | 15 | 15 | 15 | 15 | 15 | 15 | 15 |
| Min. | 5.39 | 4.34 | 5.51 | 4.62 | 5.8 | 6.06 | 2.73 | 7.68 |
| Max. | 13.08 | 11.47 | 12.7 | 11.96 | 11.71 | 12.45 | 10.76 | 13.2 |

Burst Strength: This test method was derived from the ANSI/AAMI VP20-1994 Section 8.3.3.2 and ASTM Ball Burst method D3787-01. A sample was placed on top of a circular O-ring measuring approximately 1 inch in diameter. The O-ring was seated in a grooved plate in a fixture with a hole in the middle of plate containing the O-ring. The fixture was attached to the lower jaw in a tensile tester machine. The plate with the sample was raised and clamped against an upper plate in the fixture, compressing the sample. The upper plate also contained a hole with the same diameter as the lower plate. The holes in the fixture plates are dimensioned to be just slightly larger than and to accept a rounded ball tipped rod that has a 0.38 inch diameter tip. The rod was connected to an upper jaw of the test machine that was moved down through the sample at a constant rate of 12 inches per minute. The peak load was recorded for each of fifteen samples. The average burst strength was then calculated based on the peak loads for the fifteen samples. Burst strength data is provided in Table 4, measured in pound-force (lbf).

TABLE 4

| | Pattern | | | | | | | |
|---|---|---|---|---|---|---|---|---|
| | 1 | 2 | 3 | 4 | 5 | 6 | 7 | 8 |
| Mean | 42.6 | 39.2 | 31.7 | 39 | 25.6 | 53.8 | 46.3 | 76.3 |
| Std. Dev. | 17.5 | 16.2 | 10.7 | 16.2 | 5 | 11.7 | 23.3 | 21.4 |
| N | 15 | 15 | 15 | 15 | 15 | 15 | 15 | 15 |
| Min. | 23 | 19.9 | 18.1 | 16.5 | 18.5 | 33.9 | 12 | 48.7 |
| Max. | 88.3 | 69.2 | 53.4 | 82.9 | 36.3 | 71.5 | 88.6 | 129.7 |

Tear Strength: A sample measuring approximately 2 inches×2 inches was prepared. A 1 inch slit was cut in one side (the direction to be tested) at the mid point to form two sections. One section of the sample was clamped in the lower jaw of a pneumatic fixture and the other was clamped in the top jaw of the fixture. Starting with the jaws at a minimum spacing of 1 inch, the sample was pulled at a rate of 12 inches per minute until the tear was completed. The peak force was recorded. Fifteen samples were tested and averages were then calculated. Tear strength data is provided in Table 5, measured in pound-force (lbf).

TABLE 5

| | Pattern | | | | | | | |
|---|---|---|---|---|---|---|---|---|
| | 1 | 2 | 3 | 4 | 5 | 6 | 7 | 8 |
| Mean | 4.16 | 2.52 | 2.95 | 3.56 | 3.24 | 2.98 | 3.46 | 3.87 |
| Std. Dev. | 1.24 | 0.65 | 0.8 | 0.84 | 0.87 | 0.88 | 1.43 | 1.38 |
| N | 15 | 15 | 15 | 15 | 15 | 15 | 15 | 15 |
| Min. | 2.51 | 1.33 | 1.87 | 2.08 | 2.17 | 1.82 | 2.08 | 1.74 |
| Max. | 6.85 | 3.44 | 4.25 | 5.27 | 5.64 | 5.24 | 7.2 | 7.43 |

Tensile Strength: A dog-bone shaped sample measuring approximately 1 inch×1.5 inches was placed into the pneumatic jaws of a tensile tester or equivalent device. The ends of the sample were gripped in the lower and upper jaws of the tester. The sample was pulled at a constant rate of 12 inches per minute until the sample broke. The peak load and elongation at break were recorded. The tensile strength was measured along four different directions (0° test angle, a 45° test angle, a 90° test angle and a 145° test angle) relative to the sheet of material from which the samples were taken. The tensile strength was measured in multiple directions to assess the variation in tensile strength. Fifteen samples were randomly cut for each of the four angles. The samples were tested and the averages were then calculated for each direction. Tensile strength data is illustrated in Table 6, measured in pound-force (lbf).

TABLE 6

| | 0 Degree Test Angle - Pattern | | | | | | | |
|---|---|---|---|---|---|---|---|---|
| | 1 | 2 | 3 | 4 | 5 | 6 | 7 | 8 |
| Mean | 12.79 | 16.91 | 10.74 | 13.14 | 8.61 | 18.4 | 16.57 | 21.39 |
| Std. Dev. | 3.04 | 10.21 | 3.99 | 3.43 | 1.86 | 6.43 | 9.37 | 8.79 |
| N | 15 | 15 | 15 | 15 | 15 | 15 | 15 | 15 |
| Min. | 7.6 | 7.3 | 6.5 | 8.2 | 5.7 | 11.2 | 7 | 11.6 |
| Max. | 17.5 | 47.8 | 17.1 | 18.4 | 13.4 | 35.1 | 37 | 36.1 |
| | 45 Degree Test Angle - Pattern | | | | | | | |
| | 1 | 2 | 3 | 4 | 5 | 6 | 7 | 8 |
| Mean | 15.25 | 15.94 | 11.77 | 19.23 | 10.97 | 19.85 | 16.32 | 26.1 |
| Std. Dev. | 3.83 | 7.54 | 4.22 | 4.17 | 2.76 | 7.71 | 7.37 | 9.93 |
| N | 15 | 15 | 15 | 15 | 15 | 15 | 15 | 15 |
| Min. | 9.5 | 8.9 | 7.6 | 13.4 | 5.7 | 8.9 | 7.5 | 13.4 |
| Max. | 23.8 | 35.9 | 21.3 | 28.9 | 15.4 | 37.2 | 29.2 | 48.9 |
| | 90 Degree Test Angle - Pattern | | | | | | | |
| | 1 | 2 | 3 | 4 | 5 | 6 | 7 | 8 |
| Mean | 25.21 | 20.87 | 14.17 | 21.94 | 12.71 | 24.41 | 18.25 | 34.42 |
| Std. Dev. | 10.2 | 6.85 | 6.05 | 7.51 | 4.16 | 9.71 | 6.68 | 9.65 |
| N | 15 | 15 | 15 | 15 | 15 | 15 | 15 | 15 |
| Min. | 14.2 | 9.4 | 4.9 | 8.3 | 7.5 | 13.4 | 9.1 | 15.9 |
| Max. | 54.6 | 34.2 | 24.4 | 33.5 | 21.6 | 42.3 | 37.6 | 47.2 |
| | 135 Degree Test Angle - Pattern | | | | | | | |
| | 1 | 2 | 3 | 4 | 5 | 6 | 7 | 8 |
| Mean | 17.13 | 18.01 | 11.64 | 13.96 | 12.11 | 15.75 | 15.35 | 25.19 |
| Std. Dev. | 5.67 | 6.46 | 5.14 | 4.6 | 3.44 | 4.78 | 6.44 | 8.46 |
| N | 15 | 15 | 15 | 15 | 15 | 15 | 15 | 15 |
| Min. | 9.7 | 9.2 | 5.6 | 7.9 | 7.1 | 7.9 | 5.1 | 17.4 |
| Max. | 34.2 | 34.5 | 22.1 | 23.4 | 20 | 22.8 | 27.7 | 51.9 |

Stiffness: A tensile tester in compression mode with jaw clamps at least one inch wide is used to determine the stiffness of the sample. The tensile tester measures the amount of force required to bend the sample. A 1 inch×1.5 inch sample is cut for each test angle, and the sample is positioned lengthwise in the jaws of the tensile tester with a gauge length of one inch. The stiffness is measured at four different radial directions to assess the variation in stiffness. In particular, the stiffness was measured at a 0° test angle, a 45° test angle, a 90° test angle and a 145° test angle. Three samples were tested and the averages were then calculated for each direction. Stiffness data is provided in Table 7, measured in pound-force (lbf).

TABLE 7

| | 0 Degree Test Angle - Pattern | | | | | | | |
|---|---|---|---|---|---|---|---|---|
| | 1 | 2 | 3 | 4 | 5 | 6 | 7 | 8 |
| Mean | 0.0422 | 0.0717 | 0.0594 | 0.173 | 0.0727 | 0.123 | 0.0871 | 0.195 |
| Std. Dev. | 0.0154 | 0.0473 | 0.0064 | 0.098 | 0.0242 | 0.0588 | 0.0488 | 0.0986 |
| N | 3 | 3 | 3 | 3 | 3 | 3 | 3 | 3 |

| | 45 Degree Test Angle - Pattern | | | | | | | |
|---|---|---|---|---|---|---|---|---|
| | 1 | 2 | 3 | 4 | 5 | 6 | 7 | 8 |
| Mean | 0.0798 | 0.0868 | 0.0484 | 0.1354 | 0.0828 | 0.1383 | 0.1488 | 0.1817 |
| Std. Dev. | 0.066 | 0.0473 | 0.0253 | 0.0801 | 0.0545 | 0.0258 | 0.0623 | 0.029 |
| N | 3 | 3 | 3 | 3 | 3 | 3 | 3 | 3 |
| | 90 Degree Test Angle - Pattern | | | | | | | |
| | 1 | 2 | 3 | 4 | 5 | 6 | 7 | 8 |
| Mean | 0.0581 | 0.1418 | 0.0469 | 0.0741 | 0.0806 | 0.1514 | 0.1038 | 0.2769 |
| Std. Dev. | 0.0303 | 0.0476 | 0.0209 | 0.0445 | 0.0447 | 0.0402 | 0.0301 | 0.0474 |
| N | 3 | 3 | 3 | 3 | 3 | 3 | 3 | 3 |
| | 135 Degree Test Angle - Pattern | | | | | | | |
| | 1 | 2 | 3 | 4 | 5 | 6 | 7 | 8 |
| Mean | 0.1255 | 0.1466 | 0.0666 | 0.0379 | 0.0684 | 0.1949 | 0.0724 | 0.1926 |
| Std. Dev. | 0.0797 | 0.0889 | 0.0452 | 0.0142 | 0.0387 | 0.0732 | 0.028 | 0.0946 |
| N | 3 | 3 | 3 | 3 | 3 | 3 | 3 | 3 |

It should be understood that the foregoing description of various embodiments of the invention are intended merely to be illustrative thereof and that other embodiments, modifications, and equivalents of the invention are within the scope of the invention recited in the claims appended hereto.

What is claimed is:

1. An implantable prosthesis for repairing a tissue or muscle wall defect, the implantable prosthesis comprising:
a perforated sheet of a biologically compatible material to cover the tissue or muscle wall defect, the perforated sheet having a plurality of perforations extending completely through the perforated sheet, the perforations being distributed across a substantial portion of the perforated sheet in a non-grid arrangement, each of the plurality of perforations having a diameter, each perforation being separated from an adjacent perforation by a portion of material having a length between adjacent perforations that is at least four times the diameter of the perforations.

2. The implantable prosthesis of claim 1, wherein the biologically compatible material is a natural material.

3. The implantable prosthesis of claim 2, wherein the biologically compatible material is derived from porcine dermis.

4. The implantable prosthesis of claim 1, wherein the biologically compatible material is a resorbable material.

5. The implantable prosthesis of claim 4, wherein the biologically compatible material is configured to be completely resorbed more than one year after implanted into a body.

6. The implantable prosthesis of claim 1, wherein each perforation is separated from an adjacent perforation by a portion of material having a length between adjacent perforations that is at least 0.5 inches.

7. The implantable prosthesis of claim 1, wherein the diameter of each of the plurality of perforations is substantially constant.

8. The implantable prosthesis of claim 1, wherein the diameter of each of the plurality of perforations is at least 0.093 inches.

9. The implantable prosthesis of claim 1, wherein the perforations are non-uniformly distributed across the perforated sheet in an arrangement that includes groups of perforations arranged in a plurality of concentric circular patterns about a reference point, the plurality of concentric circular patterns being spaced apart from each other by a radial distance relative to the reference point of at least four times the diameter of the perforations.

10. The implantable prosthesis of claim 1, wherein the perforated sheet of biologically compatible material has a substantially elliptical shape.

11. The implantable prosthesis of claim 1, wherein the plurality of perforations are substantially circular.

12. An implantable prosthesis for repairing a tissue or muscle wall defect, the implantable prosthesis comprising:
a perforated sheet of a biologically compatible material to cover the tissue or muscle wall defect, the perforated sheet having a plurality of perforations extending completely through the perforated sheet, the perforations being non-uniformly distributed across the perforated sheet in an arrangement that includes groups of perforations arranged in a plurality of concentric circular patterns about a reference point, each of the plurality of perforations having a diameter, the plurality of concentric circular patterns being spaced apart from each other by a radial distance relative to the reference point of at least four times the diameter of the perforations.

13. The implantable prosthesis of claim 12, wherein the biologically compatible material is a natural material.

14. The implantable prosthesis of claim 13, wherein the biologically compatible material is derived from porcine dermis.

15. The implantable prosthesis of claim 12, wherein the biologically compatible material is a resorbable material.

16. The implantable prosthesis of claim 15, wherein the biologically compatible material is configured to be completely resorbed more than one year after implanted into a body.

17. The implantable prosthesis of claim 12, wherein each perforation is separated from an adjacent perforation by a portion of material having a length between adjacent perforations that is at least 0.5 inches.

18. The implantable prosthesis of claim 12, wherein the diameter of each of the plurality of perforations is substantially constant.

19. The implantable prosthesis of claim 12, wherein the diameter of each of the plurality of perforations is at least 0.093 inches.

20. The implantable prosthesis of claim 12, wherein the perforated sheet of biologically compatible material has a substantially elliptical shape.

21. The implantable prosthesis of claim 12, further comprising a perforation positioned at the reference point.

22. The implantable prosthesis of claim 12, wherein the reference point is located at the center of the perforated sheet.

23. The implantable prosthesis of claim 12, wherein the plurality of concentric circular patterns are radially spaced apart from each other by a substantially equal distance.

24. The implantable prosthesis of claim 12, wherein the plurality of perforations are substantially circular.

25. The implantable prosthesis of claim 12, wherein the plurality of concentric circular patterns are spaced apart from each other by at least 0.5 inches.

26. An implantable prosthesis for repairing a tissue or muscle wall defect, the implantable prosthesis comprising:
a perforated sheet of a biologically compatible material to cover the tissue or muscle wall defect, the perforated sheet having a plurality of perforations extending completely through the perforated sheet, the perforated sheet including a first portion and a second portion separated by an imaginary straight line extending across the perforated sheet and through the center of the sheet, the plurality of perforations being non-uniformly distributed across a substantial portion of the perforated sheet, the plurality of perforations including a first pattern of perforations in the first portion of the perforated sheet and a second pattern of perforations in the second portion of the perforated sheet, the second pattern being different from the first pattern.

27. The implantable prosthesis of claim 26, wherein the biologically compatible material is a natural material.

28. The implantable prosthesis of claim 27, wherein the biologically compatible material is derived from porcine dermis.

29. The implantable prosthesis of claim 26, wherein the biologically compatible material is a resorbable material.

30. The implantable prosthesis of claim 29, wherein the biologically compatible material is configured to be completely resorbed more than one year after implanted into a body.

31. The implantable prosthesis of claim 26, wherein each perforation is separated from an adjacent perforation by a portion of material having a length between adjacent perforations that is at least 0.5 inches.

32. The implantable prosthesis of claim 26, wherein each of the plurality of perforations has a diameter, and each perforation is separated from an adjacent perforation by a portion of material having a length between adjacent perforations that is at least four times the diameter of the perforations.

33. The implantable prosthesis of claim 32, wherein the diameter of each of the plurality of perforations is substantially constant.

34. The implantable prosthesis of claim 32, wherein the diameter of each of the plurality of perforations is at least 0.093 inches.

35. The implantable prosthesis of claim 32, wherein the perforations are non-uniformly distributed across the perforated sheet in an arrangement that includes groups of perforations arranged in a plurality of concentric circular patterns about a reference point, the plurality of concentric circular patterns being spaced apart from each other by a radial distance relative to the reference point of at least four times the diameter of the perforations.

36. The implantable prosthesis of claim 26, wherein the perforated sheet of biologically compatible material has a substantially elliptical shape.

37. The implantable prosthesis of claim 26, wherein the plurality of perforations are substantially circular.

38. An implantable prosthesis for repairing a tissue or muscle wall defect, the implantable prosthesis comprising:
a perforated sheet of a biologically compatible material to cover the tissue or muscle wall defect, the perforated sheet having a plurality of perforations extending completely through the perforated sheet, each of the plurality of perforations having a diameter, each perforation being separated from an adjacent perforation by a portion of material having a length between adjacent perforations that is at least four times the diameter of the perforations, the perforations being distributed across the perforated sheet in an arrangement that includes groups of perforations arranged in a plurality of concentric circular patterns about a reference point, the plurality of concentric circular patterns being radially spaced apart from each other by a substantially equal distance.

39. The implantable prosthesis of claim 38, wherein the biologically compatible material is a natural material.

40. The implantable prosthesis of claim 39, wherein the biologically compatible material is derived from porcine dermis.

41. The implantable prosthesis of claim 38, wherein the biologically compatible material is a resorbable material.

42. The implantable prosthesis of claim 41, wherein the biologically compatible material is configured to be completely resorbed more than one year after implanted into a body.

43. The implantable prosthesis of claim 38, wherein each perforation is separated from an adjacent perforation by a portion of material having a length between adjacent perforations that is at least 0.5 inches.

44. The implantable prosthesis of claim 38, wherein the diameter of each of the plurality of perforations is substantially constant.

45. The implantable prosthesis of claim 38, wherein the diameter of each of the plurality of perforations is at least 0.093 inches.

46. The implantable prosthesis of claim 38, wherein the perforated sheet of biologically compatible material has a substantially elliptical shape.

47. The implantable prosthesis of claim 38, further comprising a perforation positioned at the reference point.

48. The implantable prosthesis of claim 38, wherein the reference point is located at the center of the perforated sheet.

49. The implantable prosthesis of claim 38, wherein the plurality of perforations are circular.

50. The implantable prosthesis of claim 38, wherein the plurality of concentric circular patterns are spaced apart from each other by at least 0.5 inches.

51. A method of repairing or augmenting a tissue, muscle or organ defect with an implantable prosthesis including a perforated sheet of a biologically compatible material to cover the tissue or muscle wall defect, the perforated sheet having a plurality of perforations extending completely through the perforated sheet, the perforations being distributed across a substantial portion of the perforated sheet in a non-grid arrangement, each of the plurality of perforations having a diameter, each perforation being separated from an adjacent perforation by a portion of material having a length between adjacent perforations that is at least four times the diameter of the perforations, the method comprising:
(a) positioning the implantable prosthesis so that the perforated sheet covers a defect; and
(b) securing the perforated sheet in place relative to the defect.

52. The method of claim 51, wherein the repair or augmentation is a hernia repair or augmentation procedure.

53. The method of claim 51, wherein act (b) includes fastening the perforated sheet about the periphery thereof.

54. The method of claim 53, wherein fasteners are placed approximately 1-3 cm apart about the periphery of the perforated sheet.

55. The method of claim 53, wherein the perforated sheet is secured with sutures placed about the periphery.
